(19) United States
(12) Patent Application Publication
(10) Pub. No.: US 2023/0310142 A1
(43) Pub. Date: Oct. 5, 2023

(54) RETROMAMMARY BREAST-CONSERVING SURGERY WITH DICED ACELLULAR DERMAL MATRIX FOR VOLUME REPLACEMENT IN BREAST CANCER

(71) Applicants: Jean Schneider, Houston, TX (US); L&C BIO CO., LTD., Seongnam-si (KR); The Catholic University of Korea Industry-Academic Coorporation Foundation, Seoul (KR)

(72) Inventors: Jean Schneider, Houston, TX (US); Young Jin Suh, Suwon (KR); Ye Won Jeon, Suwon (KR); Seung Taek Lim, Suwon (KR); Whan Chul Lee, Seoul (KR)

(21) Appl. No.: 17/711,202

(22) Filed: Apr. 1, 2022

(51) Int. Cl. A61F 2/12; A61B 10/02

(52) U.S. Cl. CPC A61F 2/12; A61B 10/0233; A61L 2430/04

(57) ABSTRACT

The present invention relates to breast-conserving surgery with volume replacement using dADM for breast cancer.

The surgical method according to the present invention is oncologically and cosmetically effective in terms of reducing postoperative fat necrosis, and may easily preserve the volume and shape of the preoperative breast mound. The effective shortening of the surgical time and a significant reduction in intraoperative bleeding may significantly reduce the incidence of postoperative complications such as postoperative intramammary fat necrosis and skin flap necrosis after the occurrence of subcutaneous fat necrosis.

### BACKGROUND

### 1. Field of the Invention

The present invention relates to breast-conserving surgery using a diced acellular dermal matrix (dADM).

### 2. Discussion of Related Art

Breast cancer is one of the leading causes of death for women worldwide. Recently, the age-standardized rate per 100,000 persons was 46.3 worldwide (Non-Patent Document 1). In Korea, the age-standardized rate per 100,000 persons was 59.8, which is the highest level in Asia. Traditionally, there are two major methods for removing breast cancer: one is a mastectomy which completely removes the breast, and the other is breast-conserving surgery (BCS) which removes a part of the breast in which cancer is located and the other healthy part is conserved (Non-Patent Document 2).

Although BCS has an advantage of being able to avoid a mutilating mastectomy, the immediate breast volume and shape right after BCS are asymmetrical with the contralateral healthy breast, and there is a disadvantage in that the appearance is far from beautiful.

In the decades since BCS began to be utilized to treat breast cancer, the purpose of breast cancer treatment has been to detect breast cancer as early as possible, and preserve the volume and shape of the ipsilateral breast as much as possible. This has resulted in the latest concept in oncoplastic breast cancer surgery (OPS) (Non-Patent Document 3).

In addition to a route of approach, oncologic complete removal obtains reasonable safe margins from the outermost surfaces of the resected cancer, which has to be confirmed by histologic examination. Afterward, as local treatment, external radiation therapy is performed to minimize local recurrence in the remaining breast. Accordingly, when the complete removal of resectable breast cancer is clinicopathologically ensured, the main purpose of OPS is to minimize or prevent breast deformity caused by postoperative complications such as intramammary fat necrosis close to an index tumor bed along with or without chemotherapy, and the shape and volume of the ipsilateral breast to be a cosmetically acceptable.

There are various methods to do this. First, in the case of a large breast, the typical method is the inverted T method in which a part of the breast having cancer is removed and the overall breast size along with that of the opposite breast is reduced, whereas in the case of a small breast, one of various methods is volume replacement using surrounding breast tissue. However, when the size of breasts is not large enough, a latissimus dorsi flap procedure or others using muscles, or a TRAM flap procedure or skin-sparing mastectomy of completely removing internal breast tissue while conserving most of the skin and inserting an implant thereinto or using one's own abdominal tissue is utilized. Therefore, with the exception of a method of removing cancer and suturing a wound by pulling surrounding tissue, there is no choice but to insert an implant or rebuild a breast using adjacent tissue, and in this case, either method can cause two problems. The first is that a long additional incision remains non-cosmetically, and the second is that a patient receives an unnatural result due to the shape being different from that of the patient's original breast. In most of the cases, the shape of an artificially created breast has an unnatural nonsymmetric quality.

Therefore, to achieve the original purpose of OPS, it is important to find a way to reach the goal of treating breast cancer while maintaining the shape of the breast as it was when a patient did not have breast cancer. For this purpose, attempts have been made to replace the defect of the breast having breast cancer (volume replacement), and various materials have been used, but there has always been a disadvantage of reduced patient satisfaction. The method of inserting an implant increases satisfaction with a woman who wants bigger breasts after surgery, but not every woman wants bigger breasts compared to prior to surgery, and this method requires higher cost. For this reason, few patients choose this method.

While looking for methods and materials that can supplement these shortcomings and replace them, the inventors started to pay attention to the acellular dermal matrix (ADM; Megaderm®), which is already being used in plastic surgery procedures. A method of cutting sheet-type ADM and tailoring it to a breast, or filling the folded ADM sheet in the defect remaining after the removal of breast cancer has been used, but the same problem that arose in previously reported methods using other materials has been identified. That is, a cavity generated when breast cancer is removed has an irregular shape, which is because it was difficult to transform it to have a suitable shape for a cavity needed for a standardized ADM having the same size. Therefore, new technology is needed to fill in a defect.

### SUMMARY OF THE INVENTION

The present invention is directed to providing a surgical method including breast-conserving surgery using a retromammary approach and breast reconstruction (volume replacement) using a diced acellular dermal matrix (dADM).

The present invention is also directed to providing a surgical method for reducing intramammary fat necrosis while maintaining a breast mound.

The present invention provides a surgical method for breast reconstruction, which includes:

removing a tumor by conducting breast-conserving surgery (BCS),

approaching to the lowermost breast parenchyme containing breast cancer through a retromammary plane via incision at the lateral or inferior side of breast (transaxillary or inframammary fold (IMF) route) and then removes breast cancer with safety margins upwards from the bottom to subcutaneous fat layer; and

filling a defect from which the tumor is removed with dADM.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a schematic diagram illustrating a method for breast-conserving surgery according to a conventional approach such as an anterior approach;

FIG. 2 is a schematic diagram illustrating a method for breast-conserving surgery according to the retromammary approach according to the present invention; and

FIGS. 3 to 6 are schematic diagrams illustrating retromammary approaches based on the location of a tumor.

### DETAILED DESCRIPTION OF EXEMPLARY EMBODIMENTS

The present invention relates to a surgical method for breast reconstruction, which includes removing a tumor by conducting breast-conserving surgery (BCS),

approaching to the lowermost breast parenchyme containing breast cancer through a retromammary plane via incision at the lateral or inferior side of breast (transaxillary or inframammary fold (IMF) route) and then removes breast cancer with safety margins upwards from the bottom to subcutaneous fat layer; and

filling a defect from which the tumor is removed with dADM.

Hereinafter, a surgical method for breast reconstruction according to the present invention will be described in detail.

The surgical method for breast reconstruction according to the present invention may include:
(A) removing a tumor by conducting breast-conserving surgery (BCS) (hereinafter, tumor removal step); and
(B) filling a defect from which the tumor is removed with dADM (hereinafter, defect filling step).

Step (A) in the present invention may be a tumor removal step, in which the tumor in the breast may be removed through breast-conserving surgery (BCS).

Breast-conserving surgery (BCS) is one of the breast cancer surgery methods that are available, and it involves a surgery of removing a part of the breast in which cancer is located and conserving the remaining healthy part. When the size of cancer is small compared to the overall breast size, it is possible to perform such surgery.

In the present invention, the breast-conserving surgery can be performed oncologically safe and cosmetically through a retromammary approach.

Conventionally, breast-conserving surgery is performed through an anterior approach to a tumor via a skin incision right above the tumor. However, when a defect is filled with a filler after the breast-conserving surgery upon using the anterior approach, there is an increased risk of red breast syndrome, and there is a problem in which substantially identifiable fat necrosis occurs around an incision before and after external radiation therapy. In addition, when the anterior approach is used, there is a risk of bringing about unaesthetic results due to the incision on the breast skin.

Therefore, in the present invention, the above-described problems may be overcome by performing breast-conserving surgery through a retromammary approach.

In one embodiment, in Step (A), a tumor may be removed as a column containing cancer with safety margins from the bottom to subcutaneous fat layer as upward direction through a retromammary approach via a transaxillary or inframammary fold (IMF; inframammary crease or inframammary line) route. Here, working space between pectoralis major muscles and the lowermost bottom surface of breast may refer to the retromammary space, and the inframammary fold may refer to the natural lower limit of the breast skin where the breast and the chest meet.

Specifically, the retromammary approach involves making a transaxillary or inframammary fold incision, and then entering the retromammary space from the lateral or inferior side of the pectoralis major muscle via the incision site.

In the case of the transaxillary approach, breast-conserving surgery may be performed by making a 3 to 5-cm incision in the skin fold under the armpit, and in the case of the inframammary fold approach, breast-conserving surgery may be performed by making a 3 to 5-cm incision at the lowermost limit of the breast. Both cases have an advantage of reducing the visibility of postoperative wounds and do not leave visible scars on the skin of the breast.

In one embodiment, according to the retromammary approach, dissection plane may be made anteriorly to prepectoral fascia behind an index tumor, and breast cancer may be removed with safety margins by dissecting upward (towards the skin and subcutaneous fat layer) while securing as many subcutaneous vascular network as possible overlying the breast cancer to maintain overlying skin integrity. The surgical method may easily obtain safety margins in all directions related to the index tumor.

In one embodiment, an axillary or inframammary fold incision may be used to reach an upper inner quadrant tumor.

In one embodiment, a partial mastectomy may be performed along with sentinel lymph node biopsy through an axillary incision.

In one embodiment, an incision length may vary according to a tumor size and surgical conditions.

Breast-conserving surgery in the present invention may be performed by a method known in the art, and specifically, with the exception of the approach, may be performed in the same manner as the method for breast-conserving surgery using an anterior approach.

In the present invention, after Step (A), a defect filling step (B) may be performed. In Step (B), a defect, which is a tumor-removed space, may be filled with dADM. In the present invention, this step may be expressed as volume replacement, and the defect may be expressed as a cavity.

Through the tumor removal step (A), cancer (tumor) located on virtually divided quadrants may be removed, and thus an additional step of filling in a dead space, that is, a defect, after tumor removal may be performed.

In the present invention, after the retromammary breast-conserving surgery, the defect may be filled with the dADM. In the present invention, since the dADM is filled through the incision made during the breast-conserving surgery, no additional incision of an unaesthetic nature is required for the step of volume replacement.

In one embodiment, to prevent the spillage of the filled dADM, the defect may be filled with the dADM, and then the aperture of the cylindrical cavity for dADM volume replacement may be closed using interlocking suture.

When the subcutaneous fat layer is thin, it is possible to sense the diced acellular dermal matrix on the skin, but immediately after surgery, as soon as a tissue fluid is absorbed, the dADM becomes softer. With the lapse of time, the dADM adheres to and begins to fuse with surrounding tissue, so the above problem may be slowly resolved. In the present invention, breast reconstruction using the dADM may offer convenience, and compared with implant insertion involving much higher cost and longer hospitalization, it is less expensive and requires a shorter hospitalization period and far less budget. In addition, many female patients may find it emotionally comforting to maintain a breast volume and contour similar to pre-operative conditions upon breast reconstruction according to the present invention, in comparison to an implant insertion or a flap operation. Based on the epidemiological fact that breast cancer most often occurs in those in their 40s and 50s, there is a major advantage in that breast contours of women starting menopause, in which the shape of their breasts have changed compared to when they were younger, can be maintained at an almost unchanged state before and after surgery. The recovery of pre-operative breast contour and symmetry after volume replacement using implants or local/remote flaps is not technically easy in light of the fact that the breast is comprised of soft tissue and its adipose tissue often undergoes a post-operative change in volume and shape due to fat necrosis.

In the present invention, ADM consisting of an allogeneic dermal component is a specific kind of biomaterial lacking epidermal components, but type I collagen, elastin, laminin and site-specific glycosaminoglycan are included in the extracellular matrix (Non-Patent Document 4). Such ADM may impart reduced antigenicity, which is immunologically inactive. In addition, an inherent dermal collagen component and some growth factors may be used to reinforce soft tissue repair or promote wound healing. Therefore, it is shown that the ADM becomes integrated through revascularization with surrounding human body tissues within 1 to 2 weeks after insertion (Non-Patent Document 5).

In addition to the inherent characteristics of this bio-incorporation, the ADM used herein may be sterilized by irradiation in a manufacturing process, which may not impair a collagen composition.

In the present invention, the ADM is a dermal matrix obtained from human skin by acellularization technology, and may be a commercially available diced ADM product or may be obtained by removing an epidermal layer and cells from skin tissue. (Non-Patent Document 6)

In one embodiment, dADM may be prepared by (S1) removing an epidermal layer and cells from skin tissue; and

(S2) granulating the skin tissue from which the epidermal layer and the cells are removed.

The order of Steps (S1) and (S2) is not limited, and thus may be either (S1) →(S2) or (S2)→(S1).

Step (S1) involves removing an epidermal layer and cells from skin tissue.

The skin tissue may be allogeneic or heterogenic skin tissue. The “allogeneic” refers to human, the “heterogenic” refers to a mammal, which is an animal other than a human, such as a pig, a cow or a horse. However, in order to identify the empirical efficacy of the present invention, dADM that is actually inserted into a patient may use human-derived skin tissue.

In Step (S1), a decellularization solution may be used to isolate and decellularize the epidermal layer. Decellularization refers to the removal of other cell components, with the exception of the extracellular matrix from skin tissue, for example, a nucleus, a cell membrane and a nucleic acid.

As a decellularization solution, a basic solution, and specifically, one or more selected from the group consisting of sodium hydroxide, potassium hydroxide, ammonium hydroxide, calcium carbonate, magnesium hydroxide, calcium hydroxide and ammonia may be used. In the present invention, as a decellularization solution, sodium hydroxide (NaOH) may be used.

In one embodiment, the concentration of a decellularization solution may be 0.01 to 1 M or 0.05 to 0.5 M. In the cited concentration range, it becomes easy to remove cells.

Step (S2) involves granulating the skin tissue from which an epidermal layer and cells are removed.

The granulation may be performed by physically grinding the skin tissue. The type of physical process is not particularly limited, and may be, for example, dicing with a grinder or grinding by the use of spinning balls. Alternatively, the tissue may be granulated by dicing it into square shapes with regular sizes.

In one embodiment, dADM may have various forms, for example, a spherical and polygonal shapes. Here, the spherical shape is not only limited to a true sphere, but also all non-angled forms.

In one embodiment, the average diameter of dADM may be in a range of 3 to 10 mm. Here, the diameter may refer to the length of a straight line passing through the center of the dADM.

In one embodiment, dADM may have a cube form with each side length 3 to 7 mm. Cube-form ADM may be positioned after movement within the patient's cavity, such that they can be adapted to different and irregularly shaped cavities of the patient.

Although in the case of a large mass-shaped insert, due to its size, a sensation of a foreign object is problematic from the beginning, the dADM according to the present invention move within the cavity and gradually adapt to the original breast shape of a patient, which may offset the above-mentioned problem.

In addition, since the diagonal length of the cube is approximately 8 mm (√(3)×5 mm) when the length of each side of the cube is 5 mm, more space may be filled utilizing fewer cubes, and theoretically, the space may be filled up to a degree of approximately 60% more than the actual volume. Since the volume occupied by one cube is 5 ×5 ×5 mm =125 mm³, in breast cancer staging, the use of 33 cubes is sufficient for completely filling a sphere having the maximum diameter of cancer size (T1) of 2 cm (sphere 4,186 mm³, V =(4πr3)/3), and when considering the diagonal length (8 ×8 ×8 mm =512 mm³), the sphere may be fully filled with less than 33 cubes. In many cases of breast cancer, when considering that the cancer size is 5 cm or less in diameter (sphere 65,416 mm³, V =(4πr3)/3), a defect may be sufficiently filled with 523 or fewer cubes.

Moreover, this method is able to completely and oncologically remove breast cancer while ensuring safety margins and not compromising a patient's natural breast contour.

In one embodiment, when there is an adequate retromammary fat layer, it may be left and used as the posterior wall after the removal of breast cancer, and when the layer is thin, an ADM sheet may be used to seal the posterior opening to contain dADM particles or the circumferential rim of the cavity may be subjected to a purse-string suture procedure.

In addition, when the subcutaneous fat layer on the breast skin is too thin, a thin sheet of ADM may be added just underneath the subcutaneous fat layer, and then, the cavity may be filled with dADM during volume replacement procedure. Therefore, ADM particles sticking out from the skin or the feeling of sharp points thereof may be prevented or minimized.

Particularly, the present invention is characterized by performing both breast-conserving surgery using a retromammary approach and breast reconstruction (volume replacement) using dADM.

A conventional technology of a latissimus dorsi flap procedure for breast reconstruction upon breast-conserving surgery can be selected. However, this method must be accompanied by a long and easily identifiable unaesthetic incision and functional defect at a tissue harvesting site at a harvesting site and an recipient site. In addition, the achievement of symmetry with the contralateral healthy breast is not technically easy, and the body image (the volume and shape of the breast mound) after such reconstruction may not be naturally acceptable when considering the age of patients. This may particularly be the case for women with small breasts who do not want to enlarge both breasts.

Accordingly, after breast-conserving surgery, the most ideal breast reconstruction method must maintain a natural breast contour that almost completely preserves the volume and contour of the breast prior to surgery without damaging oncological results, and preferably leaves an obscure incision which is smaller and difficult to be detected, if possible.

Until now, ADM has been widely used in the body, including breast reconstruction with artificial implant after skin-conserving mastectomy and the use as a sling support for a buttress pocket. In most cases, ADM is provided as a sheet, so it has been folded or tailored to accommodate an irregular and three-dimensional cavity.

Subsequently, dADM was designed and began to be used. The dADM is more convenient in terms of filling the cavity rather than tailoring, and may have flexibility and adaptability related to the irregular shape of the cavity as well as the original breast mound. When an implant or prosthesis is large enough to be palpable, but does not fit the shape of a patient's previous breast mound, an unstable foreign body sensation may become a problem thereof. However, the dADM according to the present invention may move around for some period of time after surgery to adjust to the shape of the previous breast mound as naturally as possible without the utilization of any specific efforts or maneuvers, and can have flexibility.

Today, breast-conserving surgery along with sentinel lymph node biopsy aims to ensure safety margins to try to reduce postoperative complications and also to be minimally invasive in all aspects without compromising oncological results.

However, the conventional approach is an anterior approach, which utilizes a method of performing breast-conserving surgery in which a vertically downward approach and dissection towards a tumor from a skin incision right above the cancer is performed, and then the defect is filled in. In this case, “red breast syndrome” or “aseptic inflammation” (subcutaneous fat necrosis without detectable microorganisms or complications accompanied by skin necrosis) may be observed at a level that is more than expected, and particularly, there is a concern that substantially identifiable surrounding fat necrosis under the incision may be observed during or upon completion of adjuvant systemic chemotherapy or irradiation. Such a problem may be reported more often in patients with small breasts and/or thin subcutaneous fat layer, particularly, in patients with cancer close to the nipple-areolar complex.

The inventors of the present invention have examined the embryological characteristics of breasts and vessels around them to examine the problems and identify the causes thereof.

Three major blood supplies provide arterial blood to mammary tissue and overlying skin (Non-Patent Document 7)

These major arteries can be easily found and managed while controlling intraoperative bleeding, but a small and valuable network supporting overlying breast skin may not be saved since there are many minute blood vessels between the skin and the parenchyma.

Damage to the blood vessels in the subcutaneous layer during dissection may cause breast fat necrosis, or in the worst cases, results flap necrosis requiring additional surgical management including flap coverage. This means that a patient does not have expected surgical results (oncoplastic outcomes), thereby missing the desired original purpose of having OPS/BCS.

Therefore, in the present invention, the principle of breast cancer removal was considered simultaneously with the embryological aspect of the breast, and it was predicted that the above problem could be almost completely solved if only breast cancer could be completely removed while obtaining safety margins without damaging the subcutaneous vascular network and skin integrity of the breast. In the case of invasive breast cancer, sentinel lymph node biopsy (SLNB) or axillary lymph node dissection (ALND) should be performed for the purpose of breast cancer staging or treatment, and to this end, an axillary incision is needed. A transaxillary approach is made only through an incision, and in this way, it is possible to enter the retromammary space from the lateral side of the pectoralis major muscle, and from here, contrary to the conventional surgical method which progresses downward from the front of the breast, it is a method of removing cancer by ascending backwards from the bottom of the breast to the upper subcutaneous fat layer, that is, from the bottom to the skin.

Using the surgical method according to the present invention, compared to the previous anterior approach, the occurrence of skin problems, which have become a concern, are very rare, but also intramammary fat necrosis, saponification or wound complications, which may occur in many cases after receiving external radiation therapy, may also be prevented in most cases. In addition, the occurrence of red breast syndrome, which is sometimes observed upon utilizing the anterior approach used by the conventional surgery, is also rare. If red breast syndrome occurs, it results from non-acceptance of tissue inserted into the patient's body due to unidentified properties thereof, so it can easily disappear simply through the removal of dADM, which can be the cause thereof.

The retromammary approach to the breast using dADM does not only offer much fewer postoperative complications, but also fully applies the principle of breast-conserving surgery ensuring sufficient safety margins in all directions along with a much shorter surgery time and not necessarily damaging the natural breast contour.

### EXAMPLES

### (1) Patient recruitment

The study protocol was reviewed and approved by the Institutional Review Board of the Catholic University of Korea (VC19OESI0079). Afterward, before starting patient accrual, the clinical trial was registered as KCT0004500 on the site of the Clinical Research Information Service of Korea, which participated as one of the primary registration authorities of the WHO International Clinical Trials Registry Platform. From August 2019 to February 2020, after providing written consent, 100 breast cancer patients aged 18 or more and less than 80 years were recruited for the clinical trial. All patients had resectable female breast cancer with no suspicious type of skin invasion, regardless of size or location of an index tumor. Exclusion criteria included the occurrence of inflammatory or infectious diseases, autoimmune diseases and blood clotting disorders within one month before surgery.

In the embodiment, dADM (Megaderm®) was used to fill the defect after removal of resectable breast cancer during BCS, clinical data was stored in a database, and written consent was obtained for this study. All procedures were conducted in accordance with the ethical standards of the institutional and/or national research committee(s), and the Declaration of Helsinki in 1964 and subsequent amendments. The inventors collected data on relevant clinical parameters including demographic data, medical history, TNM staging, tumor location, and whether chemotherapy was performed and the type thereof from all patients. After successfully registering cases as planned, when dADM was intended to be used for volume replacement after performing retromammary breast-conserving surgery, upon obtaining informed consent, 501 patients were added to the study.

### 2. Procedure

With the exception of the retromammary route of the skin incision towards the index tumor of the breast, current oncologic principles associated with the conventional breast-conserving surgery have been applied.

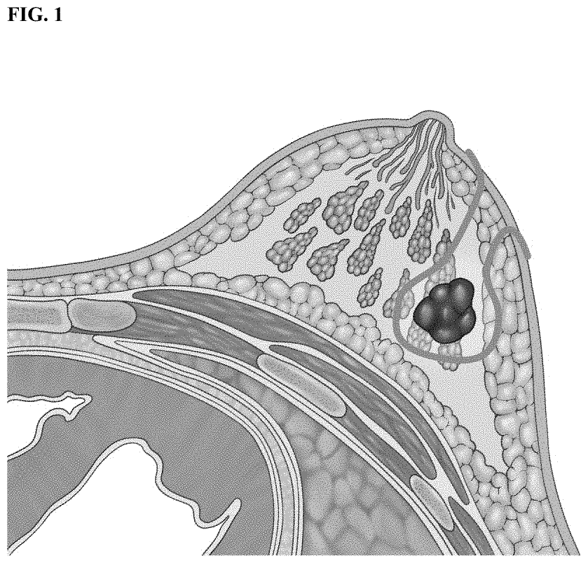

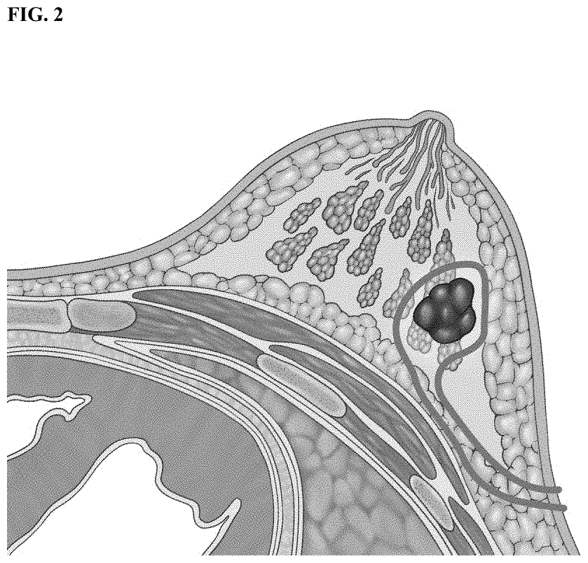

In the present invention, FIG. 1 is a schematic diagram illustrating a method for breast-conserving surgery through a conventional approach, that is, an anterior approach, FIG. 2 is a schematic diagram illustrating a method for breast-conserving surgery through the retromammary approach according to the present invention, and the breast-conserving surgery was carried out according to the method shown in the drawing.

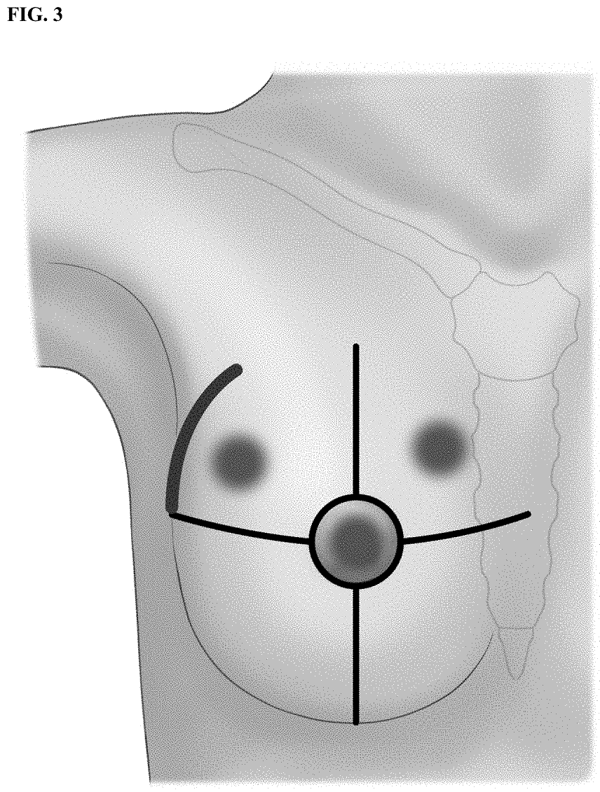

In the present invention, FIGS. 3 to 6 are schematic diagrams illustrating the method for a retromammary approach based on a tumor location. In the present invention, the location of a tumor was divided into four quadrants (i.e., upper outer, lower outer, upper inner and lower inner) based on the nipple-areolar complex, and an incision was determined based on the tumor location.

As shown in FIG. 3, when one of the tumors is located in the upper outer part, a retromammary approach via an axillary incision may be performed.

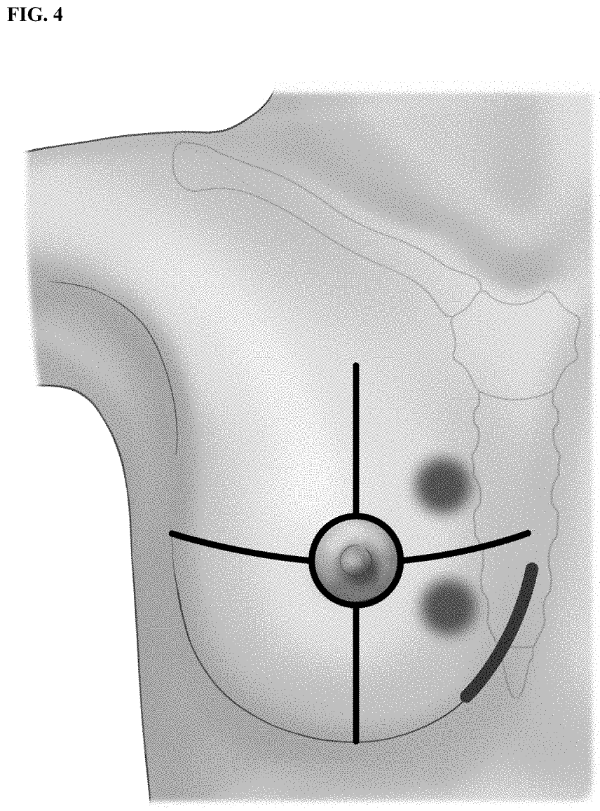

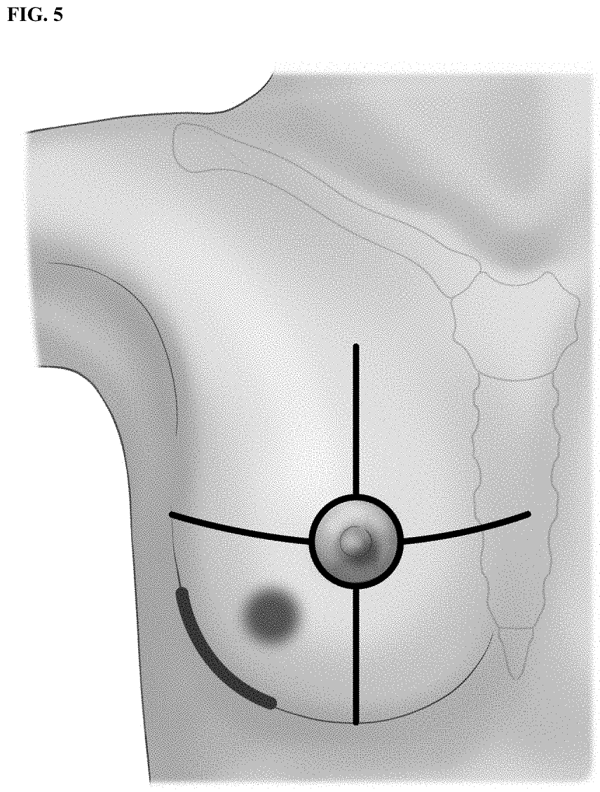

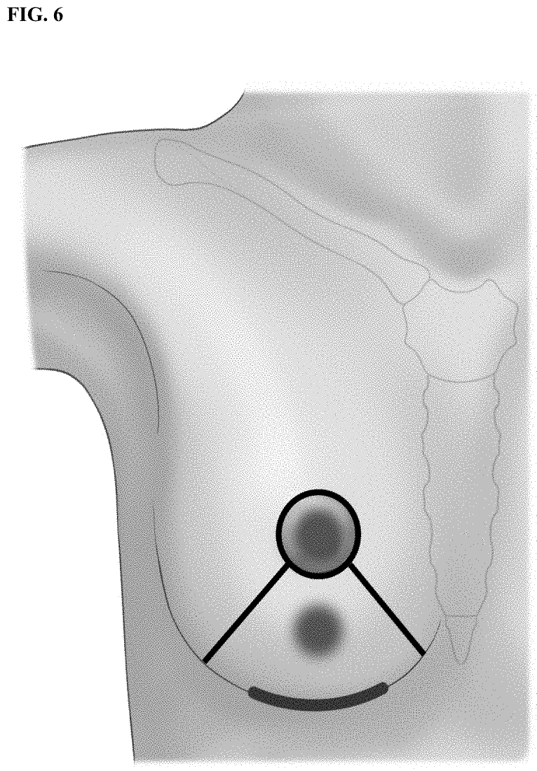

In addition, when tumors are present in the lower inner part (FIG. 4), or tumors are located in the lower part or in both the lower and central parts (FIGS. 5 and 6), a retromammary approach via a curved incision along the inframammary fold may be carried out. Specifically, in FIG. 5, tumors are located in the lower outer part, and in FIG. 6, tumors are located in the mid lower part and the center.

For example, when an index tumor was located in the upper outer or mid outer part of the nipple-areolar complex following the axillary incision, the tumor may be excised by dissecting vertically towards the subcutaneous fat layer under the skin while resecting the breast parenchyma including the index tumor and adding safety margins to a removed breast tissue column.

The index tumor located in the lower part including the lower inner or mid lower part may be directly reached via an inframammary curvilinear incision along the inframammary fold. An upper inner tumor may be reached by an axillary incision or by selectively making incisions after determination of the shorter distance from the inframammary fold. A sentinel lymph node biopsy may be performed through an integrated or separate axillary incision depending on the location of a primary incision related to the index tumor.

When the breast is too large to locate index tumor or a suspicious mass accompanied by surrounding malignant-looking microcalcifications may not be identified, image-guided needle localization may be used prior to anesthesia.

### 3. Results

Table 1 below shows the results of univariate and multivariate logistic regression analyses according to the anterior approach and posterior approach.

TABLE 1
Univariate & Multivariated logistic regression analysis (Event: RBS occurrence)

| Univariate analysis | | | | | Multivariate analysis | | | | |
|---|---|---|---|---|---|---|---|---|---|
| | | OR | 95% CI | P-value | | | OR | 95% CI | P-value |
| Approach | Anterior | 1 | | | Approach | Anterior | 1 | | |
| | Posterior | 0.351 | 0.163-0.754 | 0.007 | | Posterior | 0.426 | 0.193-0.942 | 0.035 |
| Age | | 0.947 | 0.910-0.986 | 0.008 | Age | | 0.955 | 0.918-0.994 | 0.025 |
| T stage | 0 | 1 | | | T stage | 0 | 1 | | |
| | 1 | 0.916 | 0.281-2.986 | 0.884 | | 1 | 0.866 | 0.262-2.857 | 0.813 |
| | 2 | 3.804 | 1.209-11.975 | 0.022 | | 2 | 3.466 | 1.081-11.116 | 0.037 |
| | 3 | 7.143 | 1.109-45.991 | 0.039 | | 3 | 4.778 | 0.709-32.220 | 0.108 |
| NAC | No | 1 | | | — | | | | |
| | Yes | 1.569 | 0.605-4.067 | 0.354 | | | | | |
| N stage | 0 | | | | — | | | | |
| | 1 | 1.514 | 0.544-4.213 | 0.427 | | | | | |
| | 2 | 5.802 | 1.936-17.395 | 0.002 | | | | | |
| | 3 | 4.642 | 0.934-23.072 | 0.061 | | | | | |
| BMI | | 1.105 | 1.102-1.206 | 0.025 | — | | | | |

As shown in Table 1, as a result of multivariate analysis, after making a change from the anterior approach to the method of the present invention, a posterior approach, it can be confirmed statistically that the risk (OR odd ratio) of the occurrence of complications (major =red breast syndrome including flap necrosis) is reduced by about 43%.

In addition, it can be confirmed that, in the case of utilizing the posterior approach, the risk of complications tends to decrease 0.955 based on each year of an increase in age, and as the size of breast cancer (T stage) increases, the risk of complications also tends to decrease.

Meanwhile, among 100 patients enrolled in the clinical trial according to the present invention and an additional 501 patients thereafter, only 2.3% (13 patients) had postoperative complications and thus needed surgical treatment. Except for these 13 patients, the other patients did not have severe complications after external radiation therapy and/or chemotherapy before and after surgery. In some patients, seromas were found, but only a few anecdotal aspirations were needed during an outpatient clinic visit. No hematologic malignancies were observed.

Therefore, it can be confirmed that volume replacement using dADM is a very safe and effective method that breast cancer patients who have a defect to be filled after receiving retromammary breast-conserving surgery can utilize.

The retromammary approach using volume replacement with dADM results in significantly less postoperative complications such as postoperative intramammary fat necrosis and skin flap necrosis after subcutaneous fat necrosis. These complications are very important to consider because they can lead to unaesthetic results, increased medical costs, and also dissatisfaction related to the oncoplastic principles of the breast-conserving surgery itself.

Following the retromammary approach for breast-conserving surgery, the volume replacement utilizing dADM showed promising results by reducing fat necrosis and skin problems near an incision, so it can help maintain the previous shape and volume of the breast as before surgery without anatomically and functionally sacrificing local or remote tissue collection sites which should be utilized to fill the defect after breast cancer removal.

No other significant complications associated with this procedure were found after the completion of postoperative adjuvant systemic chemotherapy, and even when wound-related problems are caused by complications associated with the hematologic toxicity of an anticancer agent during the corresponding therapeutic period, they were easily manageable via medical or surgical treatment.

The present invention provides a method for breast-conserving surgery using dADM in breast cancer.

Compared with conventional breast-conserving surgery in which an index tumor is approached by a skin incision right above the tumor, retromammary breast-conserving surgery using dADM as a filler after breast cancer removal is effective in terms of reducing postoperative fat necrosis, and can easily preserve the volume and shape of preoperative breast mound. In addition, the retromammary breast-conserving surgery can cause much less postoperative complications such as intramammary fat necrosis and skin flap necrosis after the occurrence of subcutaneous fat necrosis.

In addition, the retromammary approach can significantly reduce skin complications that can occur after surgery, including skin necrosis or intramammary fat necrosis as a result of preventing damage to tiny vascular networks under the skin.

It will be apparent to those skilled in the art that various modifications can be made to the above-described exemplary embodiments of the present invention without departing from the spirit or scope of the invention. Thus, it is intended that the present invention covers all such modifications provided they fall within the scope of the appended claims and their equivalents.

### Related Documents

1. Ferlay J, Colombet M, Soerjomataram I, Mathers C, Parkin D M, Pineros M, Znaor A, Bray F. Estimating the global cancer incidence and mortality in 2018: GLOBOCAN sources and methods. Cancer Epidemiology 2019;144(8):1941-53.
2. Ann Surg. 1894 Nov; 20(5): 497-555., Fisher B, Wolmark N, Redmond C, Deutsch M, Fisher E R. Comparison of radical mastectomy with alternative treatments. II. The clinical and biologic significance of medial-central breast cancers. Cancer 1981; 48:1863-72.
3. Weber W P, Soysal S D, Fulco I, Barandun M, Babst D, Kalbermatten D, Schaefer D J, Oertli D, Kappos E A, Haug M. Standardization of oncoplastic breast conserving surgery. Eur J Surg Oncol. 2017; 43:1236-43.
4. Tork S, Jefferson R C, Janis J E. Acellular dermal matrices: applications in plastic surgery. Semin Plast Surg 2019;33(3):173-84
5. DeGeorge B R Jr, Ning B, Salopek L S, et al. Advanced imaging techniques for investigation of acellular dermal matrix biointegration. Plast Reconstr Surg 2017;139(02):395-405
6. Lee J H, Kim H G, Lee W J. Characterization and tissue incorporation of cross-linked human acellular dermal matrix. Biomaterials 2015; 44:195-205.
7. Bland K I, Copeland E M, Klimberg V S, Gradishar W J. The Breast: Comprehensive management of benign and malignant disease. 5th. Ed. p. 146, 2018. Elsevier

1. A surgical method for breast reconstruction, comprising:
removing a tumor by conducting breast-conserving surgery (BCS);
removing the tumor upwards cylindrically from the bottom to subcutaneous fat layer with a retromammary approach through a transaxillary or inframammary fold route; and
filling a defect from which the tumor is removed with a diced acellular dermal matrix (dADM) (volume replacement).

2. The method of claim 1, wherein the retromammary approach is performed through a transaxillary or inframammary fold incision to enter the retromammary space from the lateral or inferior side of the pectoralis major muscle through the incision site.

3. The method of claim 1, wherein the diced acellular dermal matrix is filled through an incision right after removing breast cancer with safety margins during breast-conserving surgery.

4. The method of claim 1, wherein the diced acellular dermal matrix has an average diameter of 3 to 10 mm.

5. The method of claim 1, wherein the diced acellular dermal matrix is a cube having a side length of 3 to 7 mm.

6. The method of claim 1, wherein the diced acellular dermal matrix is prepared by removing an epidermal layer and cells from skin tissue; and
granulating skin tissue from which the epidermal layer and cells are removed.

7. The method of claim 6, wherein the removal of an epidermal layer and cells from the skin tissue is performed using a decellularization solution, and
the decellularization solution is one or more selected from the group consisting of sodium hydroxide, potassium hydroxide, ammonium hydroxide, calcium carbonate, magnesium hydroxide, calcium hydroxide and ammonia. therapy.

8. The method of claim 1, further comprising:
after filling with the diced acellular dermal matrix, performing radiation therapy.
